(19) United States
(12) Patent Application Publication
(10) Pub. No.: US 2007/0066855 A1
(43) Pub. Date: Mar. 22, 2007

(54) Process for the production of dimethyl ether and the co-production of H2O

(75) Inventors: Alberto Malandrino, Milan (IT); Marco Di Stanislao, Milan (IT); Giuseppe Bellussi, Piacenza (IT); Stefano Rossini, Milan (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: ENI S.p.A., Rome (IT)

(21) Appl. No.: 11/520,694

(22) Filed: Sep. 14, 2006

(30) Foreign Application Priority Data
Sep. 15, 2005 (IT) MI2005A 001700

(51) Int. Cl. C07C 41/01

(52) U.S. Cl. 568/698

(57) ABSTRACT

Process for the production of dimethyl ether and the co-production of H₂O, starting from methanol, substantially comprising a dehydration reaction of methanol carried out in a reactive distillation column with a packing of the re-active, structured and distillation type.

### BACKGROUND OF THE INVENTION

The present invention relates to a process for the production of dimethyl ether with the co-production of H₂O, starting from methanol with the use of a reactive distillation column.

DME, in general, cannot only be used as an alternative fuel, but also for feeding gas turbines and as substituents of LPG.

The known art indicates processes and technologies for the production of DME by the dehydration of methanol in vapour phase.

Methanol is produced starting from NG by means of a conventional technology; once the methanol has been produced, it reacts according to the main reaction:
2CH₃OH⇄CH₃OCH₃+H₂OΔH_{25° C.}=−23.4 KJ/mol (1w)
Reaction (1) is limited by the chemical equilibrium whose constant, in relation to the temperature, is shown in FIG. 1.

The patent U.S. Pat. No. 5,750,799 describes a process for the production of DME from methanol with different water contents in the feeding stream.

The illustrative scheme of the process is shown in FIG. 2.

The feeding of methanol (1) is mixed with the recycled product (unconverted methanol) (2), forming the stream (3) which is pumped into the pump P-1. The stream (4) is then pre-heated in the cross-flow exchanger E-1 and in the ex-changer E-2 (with HP vapour) up to the temperature of the reactor inlet R-1. The reaction is carried out in vapour phase in a catalytic fixed bed (catalyst based on alumino-silicates) at a temperature ranging from 275 to 400° C. and a pressure of about 10-15 bar.

The effluents (7) of the reactor R-1 are cooled in E-1 and E-3 (the latter exchanger is divided into two zones: the first, E-3/1, acts as the hot side of the C-2 reboiler, the second E-3/2, uses cooling water as exchange fluid) and are sent (9) to the first of the two distillation columns (C-1 and C-2) normally used in the process. The DME produced (10) is collected from the head of the first column. The second column C-2 separates the water from the unconverted methanol. The methanol recovered from the head (2) of the second column is recycled to the process, whereas the water at the bottom (12) of the column is sent to the treatment process to remove possible traces of organic substances.

The typical operating condition used so far, i.e. that when operating in gas phase, is jeopardized by a thermodynamic equilibrium, which increases with an increase in the operating temperature; it is therefore necessary to add one or more separation units to the reaction unit, for the purification of the DME produced and for the recovery of the non-reacted methanol.

The patent U.S. Pat. No. 5,684,213 describes a process for the production of DME in liquid phase, starting from methanol, in a single apparatus which combines the characteristics of a reactor and a separator: the reactive distillation column; the catalyst used for the reaction is based on zeolite, whereas the column is packed with a packing of the random type.

The apparatus used consists of a reactive section generally situated at the center of the unit, a rectification section at the top, and a stripping section at the bottom, as shown in FIG. 3; methanol is generally fed in liquid phase at the top of the reactive zone. As the catalyst used requires an operating temperature in the order of 250-300° C., the operating pressure of this column is around 40 absolute bar. Under these conditions it is not possible to obtain pure DME at the head of the column, as thermodynamic equilibrium conditions are established in the reactive zone (as can be seen below). Therefore, under the conditions described in this patent, at least one other column is necessary to separate either the DME at the top or the water at the bottom, recycling the non-reacted methanol.

### BRIEF SUMMARY OF THE INVENTION

We have now found that the energy consumptions and fixed investment costs can be reduced by carrying out the dehydration reaction of methanol in liquid phase, in a single apparatus, the reactive distillation column, using packing of the structured type.

### BRIEF DESCRIPTION OF THE DRAWINGS

FIG. 1 is a graph of the equilibrium constant (K_{eq}) for reaction (1) versus temperature.

FIG. 2 illustrates the scheme of the process described in U.S. Pat. No. 5,750,799.

FIG. 3 illustrates the apparatus described in U.S. Pat. No. 5,864,213.

FIG. 4 illustrates the scheme of the process plant according to the invention.

FIG. 5 shows a comparison of the energy performance of the process according to U.S. Pat. No. 5,750,799 (Conventional process) and the process of the invention.

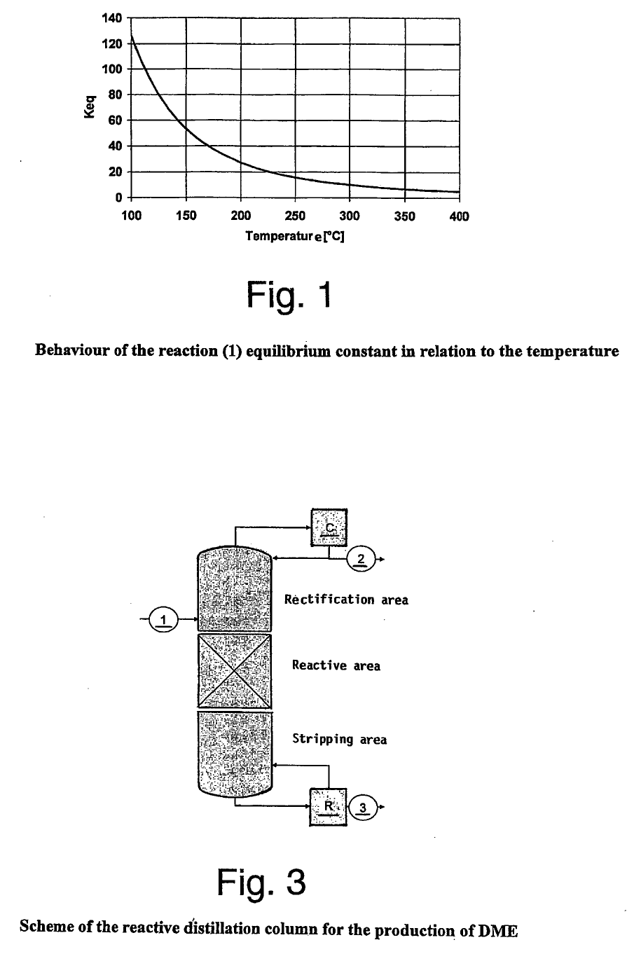

### DETAILED DESCRIPTION OF THE INVENTION

The process, object of the present invention, for the production of dimethyl ether with the co-production of water starting from methanol, substantially comprises a dehydration reaction of methanol in the presence of a suitable catalyst, carried out in a reactive distillation column having a reactive area, a rectification area and a stripping area, characterized in that it utilizes packing of the reactive and distillation type.

A packing of the reactive structured type is used for the reactive area, packed with the suitable catalyst, whereas a distillation structured packing is used for the other two areas.

The dehydration reaction in the reactive column is preferably effected at a temperature ranging from 120 to 185° C. and at a pressure ranging from 10 to 35 absolute bar, and more preferably at a temperature ranging from 120 to 145° C. and at a pressure ranging from 10 to 12 absolute bar, or at a temperature ranging from 140 to 185° C. and a pressure of between 15 and 30 absolute bar.

The catalyst used is preferably selected from a sulphonic resin or alumina or a compound based on alumina.

The reactive distillation column preferably consists of a reactive section generally situated in the center of the unit, a rectification section at the top and a stripping section at the bottom.

The process for the production of dimethyl ether in particular comprises pumping methanol, by means of a pump, and subsequently feeding said methanol to the reactive distillation section, obtaining, at the head, a mixture which is extremely rich in DME and, at the bottom, a mixture extremely rich in water.

With the help of FIG. 3, the operations effected in said reactive distillation column are described.

The liquid methanol is fed into the top of the column (1), near the area containing the catalysts. Both the reaction (in liquid phase)and the phase equilibrium—which removes the DME formed by transferring it to vapour phase, thus moving the composition of the reaction products beyond equilibrium—take place in the reactive area. At the head of the column there is a mixture which is extremely rich in DME (stream 2), whereas at the tail of the column there is a mixture which is extremely rich in water (stream 3). If the methanol conversion is total, then the separation between head and tail is practically complete.

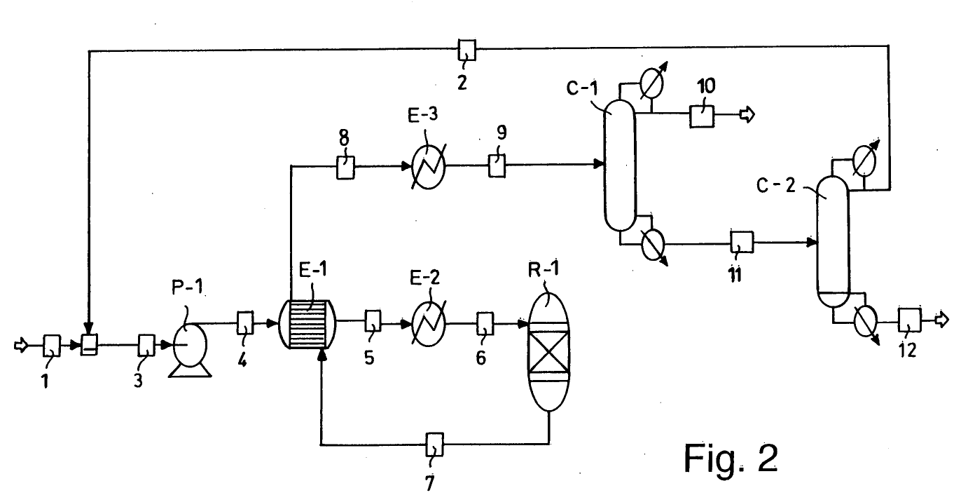

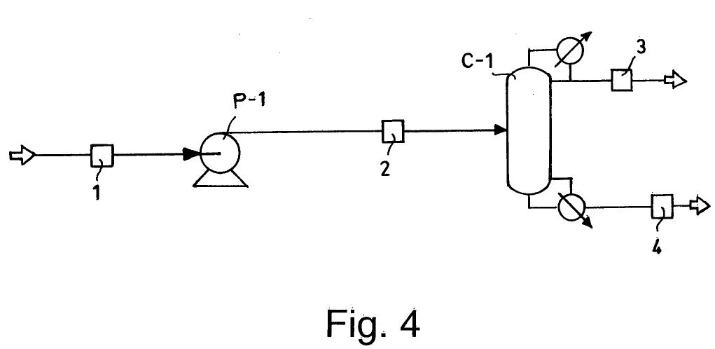

The scheme of the process plant according to the invention is preferably that shown in FIG. 4, in which the plant simplification with respect to a conventional plant (such as that shown in FIG. 2) is immediately evident.

The methanol feed (1) is pumped by means of the pump P-1 at the pressure of the column C-1. This column has a central reactive area in which the column packing and the reaction catalyst are both present. The minimum pressure value of the column is 10 bar: this value is established by the fact that a stream of practically pure DME condenses at a temperature of 45° C. and this temperature is the minimum compatible with a condenser cooled with tower water entering at a typical temperature of 30° C. The maximum value, on the other hand, is determined, for the system under examination (water-methanol-DME) by thermodynamic factors and is equal to about 30-35 bar: over this value, in fact, the volatility of the DME (whose value decreases with an increase in the pressure) decreases to such a value that the DME produced by the reaction is not capable of being transferred from liquid to vapour phase; consequently, chemical equilibrium conditions are formed in the reactive area of the column.

Two examples are shown hereunder, one of which comparative, according to the process of the known art described in U.S. Pat. No. 5,750,799, the other one according to the invention.

### EXAMPLE 1

### Comparative

According to U.S. Pat. No. 5,750,799.

With reference to FIG. 2, the methanol feed (1) is mixed with the recycled product (unconverted methanol) (2), the stream (3) formed is pumped up to 10 absolute bar into P-4. The stream 4 is then pre-heated in the cross-flow exchanger E-1 and in the exchanger E-2 (with HP vapour) up to the inlet temperature of the reactor, equal to 270° C. The reaction is carried out in vapour phase in a catalytic fixed bed (the catalyst is based on alumino-silicates) at a temperature which varies along the reactor from 275 to 400° C. and at a pressure of about 10 bar. The conversion per passage is 80%.

The reactor effluents are cooled in E-1 and E-3 to 75° C. and sent (9) to the first of the two distillation columns normally used in the process. The pressure of the first column C-1 is 10.7 bar and the reflux ratio is 0.3. The DME produced (10) is collected from the head of the first column (stream 10). The second column C-2 (P=1 bar, R=1.0) separates the water from the unconverted methanol. The methanol recovered from the head (2) of the second column is recycled to the process, whereas the water at the bottom of the column (stream 12) is sent to the treatment process to remove possible traces of organic substances.

The material balance for a production of 3,600 tpd of DME is shown in Table 1, whereas the thermal charges of the equipment for the thermal exchange of this scheme are summarized in Table 2.

TABLE 1
Material balance of the conventional process (flow-rates in tpd)

| | Stream | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| MEOH | 5007 | 1233 | 6240 | 6240 | 6240 | 6240 | 1248 | 1248 | 1248 | 14 | 1234 | 0 |
| W | 6 | 16 | 21 | 21 | 21 | 21 | 1425 | 1425 | 1425 | 0 | 1425 | 1409 |
| DME | 0 | 36 | 36 | 36 | 36 | 36 | 3624 | 3624 | 3624 | 3589 | 36 | 0 |
| Total | 5013 | 1284 | 6297 | 6297 | 6297 | 6297 | 6297 | 6297 | 6297 | 3603 | 2694 | 1409 |
| T, ° C. | 38 | 49 | 40 | 41 | 142 | 270 | 386 | 130 | 75 | 48 | 153 | 101 |
| P, bar | 1 | 1 | 1 | 11.2 | 11.2 | 11.1 | 10.9 | 10.9 | 10.9 | 10.7 | 10.7 | 1 |

TABLE 2
Thermal charges of the exchange equipment
of the conventional process

| | Thermal charge, | |
|---|---|---|
| Exchanger | MW | Utility for the exchange |
| Reboiler C-1 | 28.3 | Vapour at 11.2 bar (T = 185° C.) |
| Condenser C-1 | 20.7 | Cooling water (ΔTₘₗ = 14° C.) |
| Reboiler C-2 | 9.4 | Vapour at 11.2 bar (T = 185° C.) |
| Condenser C-2 | 35 | Cooling water (ΔTₘₗ = 20° C.) |
| E-2 | 45 | Vapour at 103 bar (T = 312° C.) |
| E-3/2 | 31 | Cooling water (ΔTₘₗ = 60° C.) |

### EXAMPLE 2

With reference to FIG. 4, the methanol feed (1) is pumped by means of P-1 from a pressure of 1 bar to the pressure of the column C-1. This column has a central reactive area in which the packing of the column itself is coupled with the reaction catalyst (sulphonic resin). The minimum value of the column pressure is equal to 10 absolute bar: this value is imposed by the fact that a stream of practically pure DME condenses at a temperature of 45° C. and this temperature is the minimum value compatible with a condenser cooled with tower water entering at a typical temperature of 30° C. The maximum value, on the other hand, is determined, for the system under examination (water-methanol-DME), by thermodynamic factors and is equal to about 30-35 absolute bar.

The pressure of the column ranges from 10 to 12 absolute bar.

The rectification and adsorption areas are also packed with structured material.

Simulations with ASPEN™ 12.1 were effected to verify the performance of a reactive column in relation to the pressure, assuming that a commercial sulphonic resin (Amberlyst 35 wet) is used as catalyst (example 2). The kinetics for the sulphonic resin was obtained from literature (Weizhu An, Karl T. Chuang and Alan R. Sanger “Dehydration of Methanol to Dimethyl Ether by Catalytic Distillation”, The Can. J. of Chem. Eng. 2004, 82., 948-955). In this work, simulations were also effected with ASPEN™ to verify the influence of the operative parameters, but no mention was made of the type of packing (and therefore to the fluid dynamics of the column).

The column is equipped with 30 theoretical plates, including a condenser and reboiler, with the feed situated at plate number 8, the reflux being fixed at 2. In the case of the sulphonic resin, the reactive area starts from plate number 10 and ends at a plate depending on the pressure of the column: this is effected to control the temperature in the reactive area and to prevent the catalyst temperature from exceeding the maximum value (about 150° C.). The volume of the reactive column is selected so as to have a molar fraction of DME at the outlet equal to 0.994.

The results of the simulation for a production of 3,600 tpd of DME are indicated hereunder. In Tables 3 and 4 the material balances and thermal charges are specified of the exchange equipment for an operating pressure of 10 absolute bar, whereas Tables 5 and 6 indicate the material balances of the exchange equipment for an operating pressure of 12 absolute bar.

TABLE 3
Material balance of the process using sulphonic resin as
catalyst (P = 10 absolute bar, flow-rates in tpd)

Stream

| | 1 | 2 | 3 | 4 |
|---|---|---|---|---|
| MEOH | 5007 | 5007 | 14 | 3 |
| W | 6 | 6 | 0 | 1406 |
| DME | 0 | 0 | 3584 | 0 |
| Total | 5013 | 5013 | 3598 | 1409 |
| T° C. | 38 | 39 | 45 | 183 |
| P, absol. bar | 1 | 12 | 10.0 | 10.7 |

TABLE 4
Thermal charges of the exchange equipment of the process
using sulphonic resin as catalyst (P = 10 abs. bar)

| Exchanger | Duty, MW | Utility for exchange |
|---|---|---|
| Reboiler C-1 | 49.6 | Vapour at 18 bar (T = 208° C.) |
| Condenser C-1 | 48.3 | Cooling water (ΔTₘₗ = 10° C.) |

| | |
|---|---|
| Reactive area from | Catalyst V 207 m³ |
| plate 10 to plate 23 | |

TABLE 5
Material balance of the process using sulphonic resin as
catalyst (P = 12 absolute bar, flow-rates in tpd)

Stream

| | 1 | 2 | 3 | 4 |
|---|---|---|---|---|
| MEOH | 5007 | 5007 | 14 | 3 |
| W | 6 | 6 | 0 | 1406 |
| DME | 0 | 0 | 3584 | 0 |
| Total | 5013 | 5013 | 3598 | 1409 |
| T° C. | 38 | 39 | 52 | 190 |
| P, absol. bar | 1 | 14 | 12.0 | 12.7 |

TABLE 6
Thermal charges of the exchange equipment of the process
using sulphonic resin as catalyst (P = 12 abs. bar)

| Exchanger | Duty, MW | Utility for exchange |
|---|---|---|
| Reboiler C-1 | 49.3 | Vapour at 21 bar (T = 215° C.) |
| Condenser C-1 | 46.6 | Cooling water (ΔTₘₗ = 17° C.) |

| | |
|---|---|
| Reactive area from | Catalyst V 140 m³ |
| plate 10 to plate 18 | |

Operating at a pressure of 12 absolute bar allows a saving of both the catalyst volume and water consumption; with respect to the latter, the saving is double: the duty is reduced (from 48.3 to 46.6 MW), and, above all, the ΔTₘ₁ increases from 10 to 17° C.

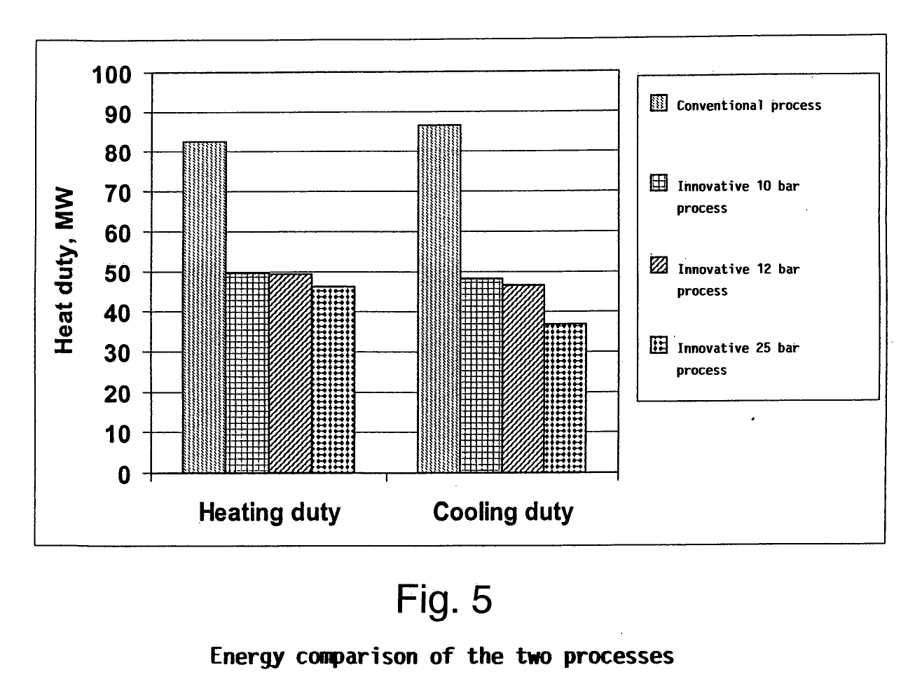

The diagram of FIG. 5 shows a comparison between the energy performances of the two processes.

On the basis of these considerations, the advantage of carrying out the operation in a reactive column is evident: with the same purity of the product obtained, there is a saving of 45% in terms of cooling duty and of 40% in terms of heating duty: this certainly leads to a saving in the operating costs.

On the basis of the above considerations, the result is that, for a pressure of 10 absolute bar, the upper rectification area requires 8 theoretical plates, the reactive area 14 and the lower stripping area 6 plates; on the contrary, for a pressure of 12 absolute bar, the corresponding numbers of plates are 8, 9 and 11.

By evaluating the heights of the theoretical plates, with packing of the structured and random type, using literature correlations (from the book of R. Taylor and R. Krishna “Multicomponent Mass Transfer”, John Wiley & Sons, Inc, 1993, pages 348-358, for packing of the random type and structured non-reactive type; and from Hoffmann et al. “Scale-up of reactive distillation column with catalytic packing”, Chem. Eng. and Proc. 43, pages 383-395, 2004, for reactive packing of the structured type) and applying it to the case under examination of the dehydration of methanol, the following average results are obtained:

TABLE 7
Comparison of the theoretical plate
heights for two types of packing

| HETP, m | Reactive area | Distillation area |
|---|---|---|
| Random packing | 0.29 | 0.32 |
| Structured packing | 0.09 | 0.11 |

It can be seen from this table that the HETP (Height Equivalent to a Theoretical Plate) for a random packing is about 3 times larger than that relating to a structured packing: this means that, with the same operating conditions, the height of a column equipped with structured packing (both in the reactive and distillation area) can be even three times smaller than the height of a column equipped with random packing. This means a considerable reduction in the investment cost.

1. Process for the production of dimethyl ether and the co-production of H₂O, starting from methanol, substantially comprising a dehydration reaction of methanol, in the presence of a suitable catalyst, carried out in a reactive distillation column (column reactor) having a reactive area, a rectification area and a stripping area, characterized in that it uses packing of the structured reactive and distillation type.

2. The process according to claim 1, wherein, in the reactive area, a packing is used of the reactive structured type, packed with the suitable catalyst, whereas in the other two rectification and stripping areas, a distillation structured packing is adopted.

3. The process according to claim 1, wherein the dehydration reaction in the reactive column is carried out at a temperature ranging from 120 to 185° C. and a pressure ranging from 10 to 35 absolute bar.

4. The process according to claim 3, wherein the temperature ranges from 120 to 145° C. and the pressure from 10 to 12 absolute bar.

5. The process according to claim 3, wherein the temperature ranges from 140 to 185° C. and the pressure from 15 to 30 absolute bar.

6. The process according to claim 1, which comprises the pumping, by means of a pump, of methanol and subsequently feeding said methanol to the reactive distillation column, obtaining, at the head, a mixture extremely rich in DME and at the tail, a mixture extremely rich in water.

7. The process according to claim 1, wherein the reactive distillation column essentially consists of a reactive area situated in the center, a rectification area at the top and a stripping area at the bottom.

8. The process according to claim 1, wherein the catalyst is a sulphonic resin or alumina or based on alumina.
